(19) United States
(12) Patent Application Publication
(10) Pub. No.: US 2018/0104463 A1
(43) Pub. Date: Apr. 19, 2018

(54) ROLL-ON DISPENSER FOR MEDICAMETS

(71) Applicant: Heinz Weber, Beaconsfield, CA (US)

(72) Inventor: Heinz Weber, Beaconsfield, CA (US)

(21) Appl. No.: 15/732,656

(22) Filed: Dec. 8, 2017

Related U.S. Application Data
(63) Continuation-in-part of application No. 14/756,372, filed on Sep. 3, 2015, now abandoned.

(51) Int. Cl. A61M 35/00; A45D 34/04

(52) U.S. Cl. CPC A61M 35/003; B65D 41/0421; A45D 34/041

(57) ABSTRACT

A container having a neck surrounding a dispensing outlet, a ball rotatably mounted and restrained in the neck, a valve seat formed within the neck, an outer wall of the neck having an upper portion and a lower portion with the upper portion of the outer wall having threads formed thereon and a lower portion having a locking mechanism formed thereon, a cap with screw threads formed on an upper portion thereof and a locking member formed on a lower portion of the cap side wall. The dispenser is childproof and ensures that the cap is sealed with the neck when closed.

The present application is a continuation-in-part of application Ser. No. 14/756,372 filed Sep. 3, 2015, the teachings of which are hereby incorporated by reference.

### FIELD OF THE INVENTION

The present invention relates to a dispenser and more particularly, relates to a roll-on dispenser suitable for dispensing a medicament on a surface such as the skin of a person.

### BACKGROUND OF THE INVENTION

Roll-on dispensers are well known in the art and are widely used for such things as deodorants and the like. A typical roll-on dispenser will include a container for the material to be dispensed with the container having a neck portion which is designed to retain a ball. A ball is mounted within the neck portion and retained in position with a relatively limited movement. A cap is usually provided for covering the neck when the roll-on dispenser is stored.

The use of roll-on dispensers has also been suggested for medical purposes. Thus, certain medications are applied to the skin of the patient for the treatment of conditions such as arthritis, gout, joint pain, etc. For such medications, the requirements for a roll-on dispenser include being child proof and for securely sealing the ball to prevent leakage during travel and the like.

### SUMMARY OF THE INVENTION

It is an object of the present invention to provide a roll-on dispenser suitable for dispensing medications on the skin of a patient.

According to one aspect of the present invention, there is provided a roll-on dispenser comprising a container, the container having a neck surrounding a dispensing outlet, a ball rotatably mounted in and retained in the neck, a value seat formed within the neck, an outer wall of the neck having an upper portion and a lower portion, the upper portion of the outer wall having threads formed thereon, the lower portion of the outer wall having a locking mechanism thereon, and a cap, the cap having a cap side wall and a cap top wall, screw threads formed on an upper portion of the cap side wall and a locking member formed on a lower portion of the cap side wall, a protrusion formed on an inner surface of the cap top wall, the protrusion engaging the ball when the cap is tightened on the container.

The roll-on dispenser, as above mentioned, comprises a container which has molded body having a central body portion and a neck. The neck includes a mouth and a ball is mounted such that a portion of the ball extends exteriorly of the mouth of the container. The ball is typically retained within the neck by a lip portion and is mounted on a valve seat.

The ball, although it may be of any conventional size, is typically a relatively small size ball having a diameter in the range of one inch. As aforementioned, the neck of the container contains a valve seat and the distance between the valve seat and the lip is designed to permit the fluid to move past the valve seat as the ball is rotated and to be dispensed. This arrangement is well known in the art.

The neck of the container is different from conventional roll-on dispensers in that it is provided with an upper neck portion and a lower neck portion. The upper neck portion, on the outer wall thereof, has screw threads which are designed to mate with screw threads on the cap. The lower portion of the neck wall contains a locking mechanism which renders the container childproof.

The screw threads are arranged so that they are a double start type first and second threads, each extending slightly more than half the circumference of the outer wall of the neck. They have a relatively high pitch for reasons which will become apparent hereinbelow.

The locking members on the lower outer wall of the neck are preferably arranged so that there are two such members diametrically opposed. However, it is within the scope of this invention that a single locking member be utilized.

In the preferred embodiment, the locking members comprise a first wall extending outwardly from the lower outer wall of the neck and a second wall extending from the free end of the first wall to merge with the lower outer wall of the neck. As such, the second wall serves as a ramp to permit a like locking member on the cap wall to slide thereover and engage with the first wall.

The first wall extends outwardly so as to provide a sharp angle with respect to the neck lower wall. The first wall preferably forms an angle of 90° or less with respect to the neck lower wall.

The locking member is also defined by a top wall sloping downwardly and outwardly from the neck lower wall. This arrangement provides for removal of the cap in an efficient manner.

The cap includes an outer side wall and a top wall. The outer side wall is preferably divided into two sections—a larger lower wall and a smaller diameter upper wall. Preferably, both surfaces of the outer wall are knurled in nature to assist in gripping. In addition to the above, preferably the side wall and the top wall contain indicia indicating how to remove the cap.

The top wall is preferably relatively small in diameter. In order to achieve this, the upper portion of the upper wall preferably has a stepped configuration to provide a relatively small upper surface. This smaller diameter upper wall allows for a less thick wall which still achieves the desired standards. On the lower surface of the top wall, there is provided a protrusion which is designed to engage the ball and press the same against the valve seat when the top is engaged with the container.

The upper inner wall will contain screw threads mating with those on the upper side wall of the container. The lower interior wall will preferably have a pair of locking members to engage with locking members on the lower outer wall of the neck of the container. Again, the locking member may take the form of a rib like member which is angled with respect to the side wall.

### BRIEF DESCRIPTION OF THE DRAWINGS

Having thus generally described the invention, reference will be made to the accompanying drawings illustrating an embodiment thereof, in which:

FIG. 1 is a side elevational exploded view of one embodiment of the roll-on dispenser of the present invention;

FIG. 2 is an elevational exploded view of the roll-on dispenser of FIG. 1 rotated through 90°;

FIG. 3 is a side elevational view with the ball in position of the roll-on dispenser;

FIG. 4 is a perspective view thereof;

FIG. 5 is a perspective view of the dispenser with the ball removed;

FIG. 6 is a further perspective view thereof;

FIG. 7 is a perspective view thereof illustrating the bottom portion of the roll-on dispenser;

FIG. 8 is a perspective view of the cover of the roll-on dispenser;

FIG. 9 is a plan view of the interior of the cover;

FIG. 10 is a sectional exploded view of the roll-on dispenser; and

FIG. 11 is a longitudinal sectional view of the assembled roll-on dispenser.

FIG. 12 is a bottom perspective view of a preferred embodiment of a cap according to the present invention;

FIG. 13 is a top perspective view thereof;

FIG. 14 is a side elevational view thereof;

FIG. 15 is a bottom plan view thereof; and

FIG. 16 is a top plan view thereof.

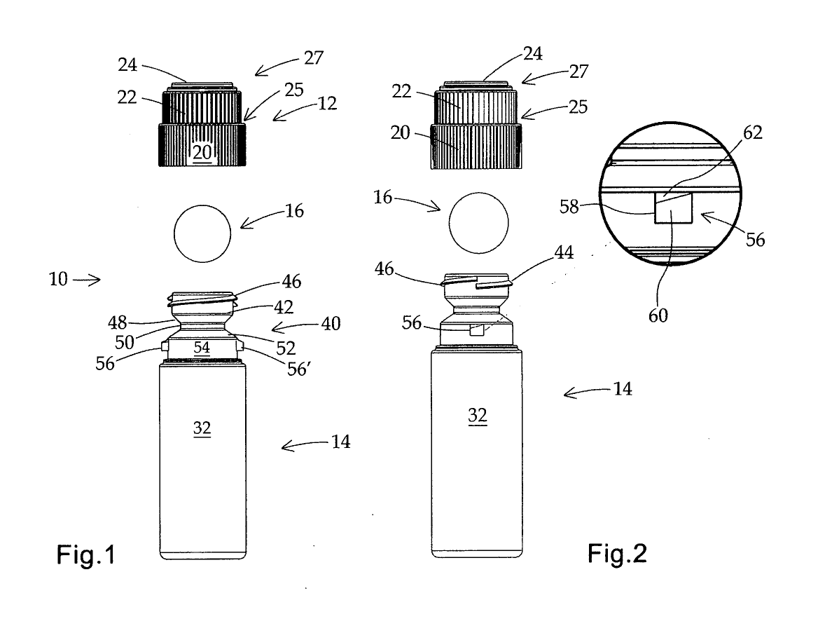

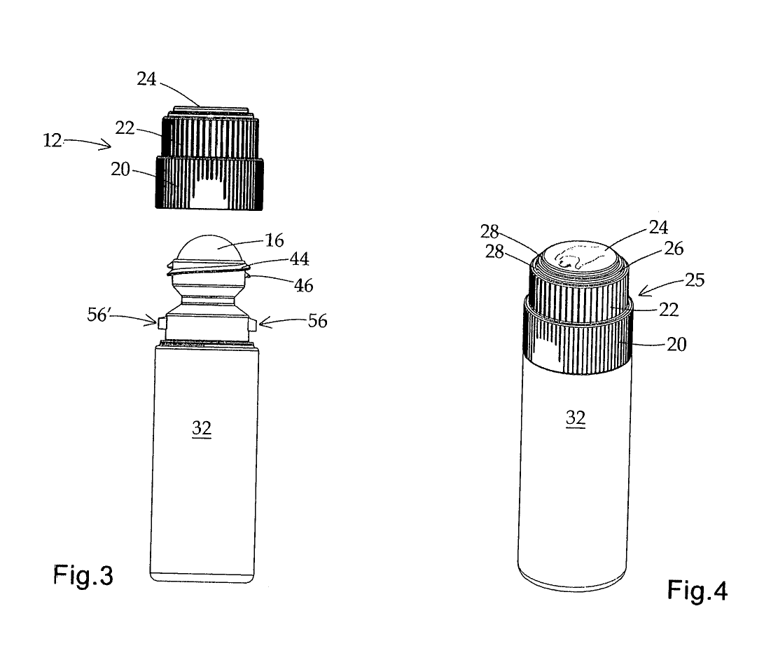

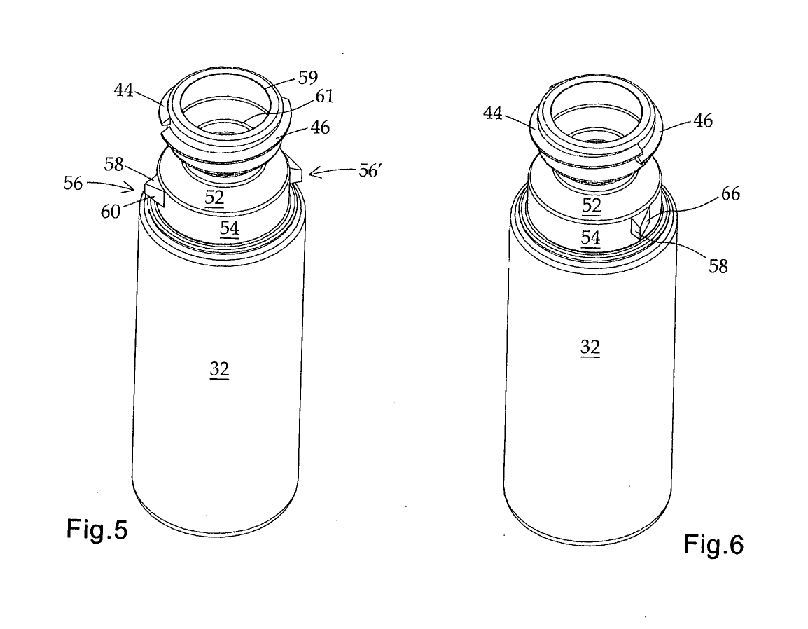

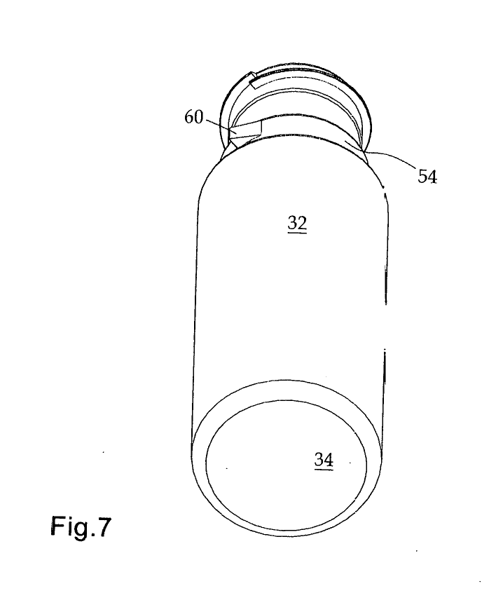

### DETAILED DESCRIPTION OF THE INVENTION

Referring to the drawings in greater detail and by reference characters thereto, there is illustrated a roll-on dispenser which is generally designated by reference numeral 10. Roll on dispenser 10 includes a top generally designated by reference numeral 12, a main body generally designated by reference numeral 14 and an applicator roller or ball generally designated by reference numeral 16.

Top or cap 12 has, as may be seen in the drawings, a lower side wall 20 and an upper side wall 22. Cap 12 also includes a top wall 24.

Intermediate upper side wall 22 and lower side wall 20 is a stepped portion generally designated by reference numeral 25. A further stepped portion 27 extends between top wall 24 and upper side wall 22 and includes a plurality of vertical wall segments 26 and a plurality of horizontal wall segments 28 which form the step pattern.

Formed on the under side of top wall 24 is a protrusion 30 which has a annular ring configuration for reasons which will become apparent hereinbelow.

Main body 14 is defined largely by a cylindrical side wall 32 and a bottom wall or base 34. At its upper end, main body 14 has a neck portion generally designated by reference numeral 40. Neck portion 40 includes an upper side wall 42 and a lower side wall 54.

Upper side wall 42 has first and second threads 44, 46 formed on an exteriorly facing surface thereof. Each thread 44, 46 extends approximately half of the circumference of upper side wall 42. Upper side wall 42 merges with an inwardly tapering wall 48 which in turn meets an intermediate vertical wall segment 50.

Situated below intermediate vertical wall segment 50 is an outwardly tapering wall 52 which meets lower vertical side wall 54. A pair of locking members 56 and 56′ are formed on the exterior surface of lower side wall 54. As will be seen, locking member 56 has a first wall 58 which extends outwardly from lower side wall 54. Preferably, first wall 58 is substantially perpendicular or forms an acute angle with lower side wall 54. Second wall 60 extends from the end of first wall 58 and merges with lower side wall 54. Second wall 60 thus forms a ramp for a further locking member (to be discussed hereinbelow) to slide along. A top wall 62 is angled downwardly from lower side wall 54. This helps to ensure a clearance of a further locking member when opening the top.

Interiorly of neck portion 40, there is provided a circular valve seat 61 which is designed to receive roller or ball 16. A lip 59 extends about the mouth of neck portion 40. In the manufacture, roller or ball 16 is inserted through the opening and is retained in position by lip 59. The insertion of roller 16 is accomplished with the roller 16 being of a harder material such as polypropylene than the container 14 which may be formed of a polyethylene material.

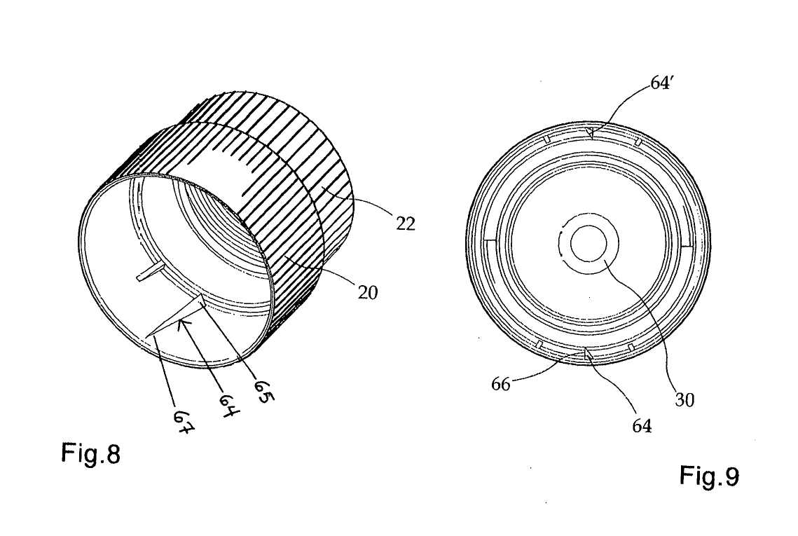

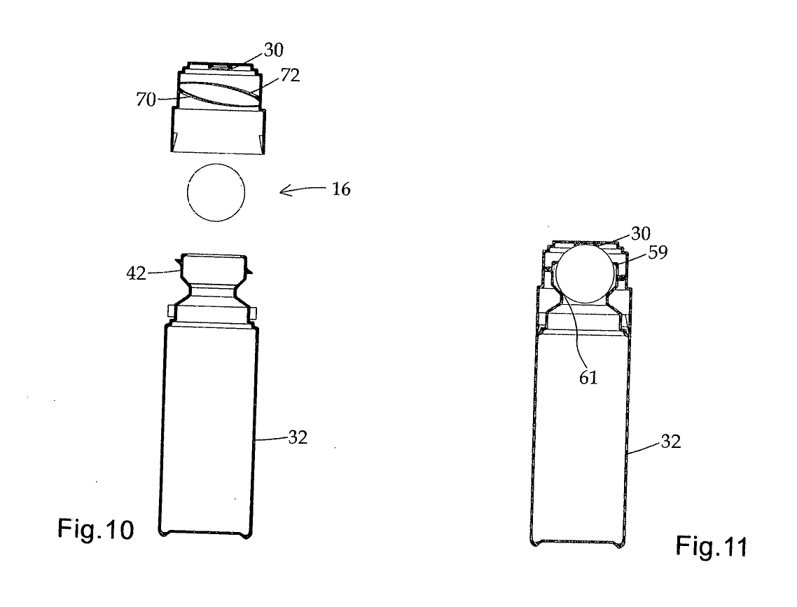

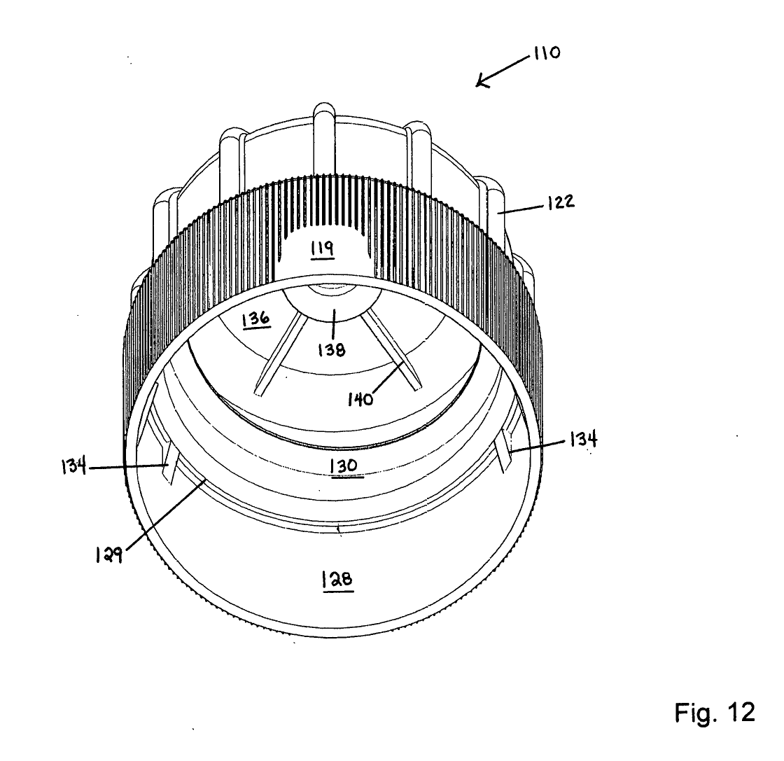

Referring back to the top 12, it will be seen that the interior is provided with a pair of locking members 64, 64′. Locking member 64 has an acute angled wall 66; locking members 64, 64′ are designed to engage with locking members 56, 56′. As seen in FIG. 8, locking member 64 extends the complete length of lower side wall 20 and also has a tapering height with the greatest height being adjacent upper side wall 22 and the smallest height being at the end of lower side wall 20.

A plurality of reinforcing ribs 68 are provided on the interior of lower side wall 20. Threads 70, 72 are designed to engage with threads 44, 46.

It will be seen that both side wall 20 and upper side wall 22 are provided with a knurled surface 74. Lower side wall 20 contains a pair of indicia 76 indicating where to push on the wall to disengage the locking members 64, 64′. A further indicia 78 is provided on the top wall 24 also indicating the action to be taken to remove the top.

As is conventional, the roller 16 and neck 40 are dimensioned so as to provide a small gap between roller 16 and valve seat 61 to permit application of the material within body 14. During transport, inner ring 30 will force roller 16 into a tight seal with valve seat 61. The locking action of the cap with the neck of the container will prevent accidental loosening of the same.

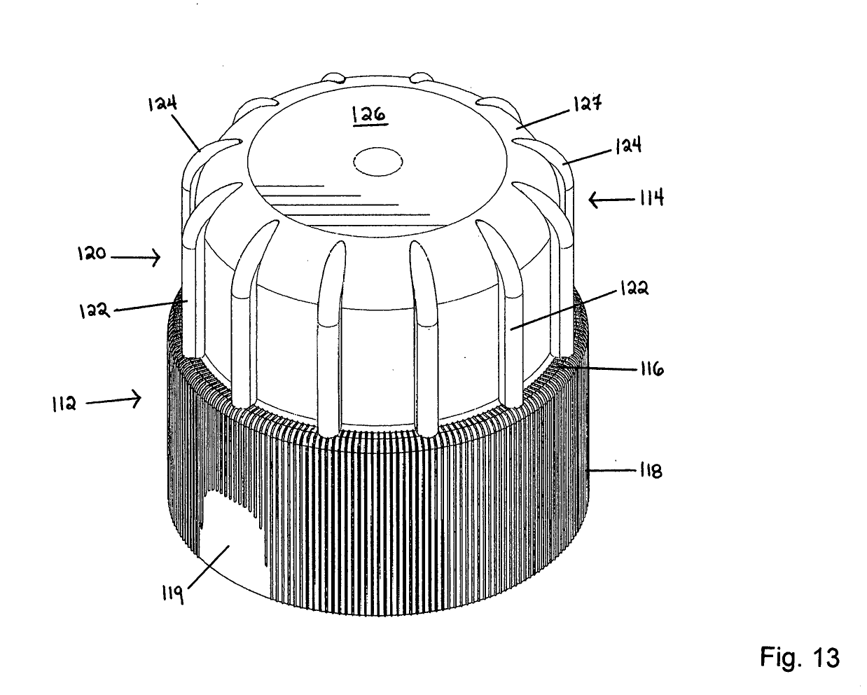

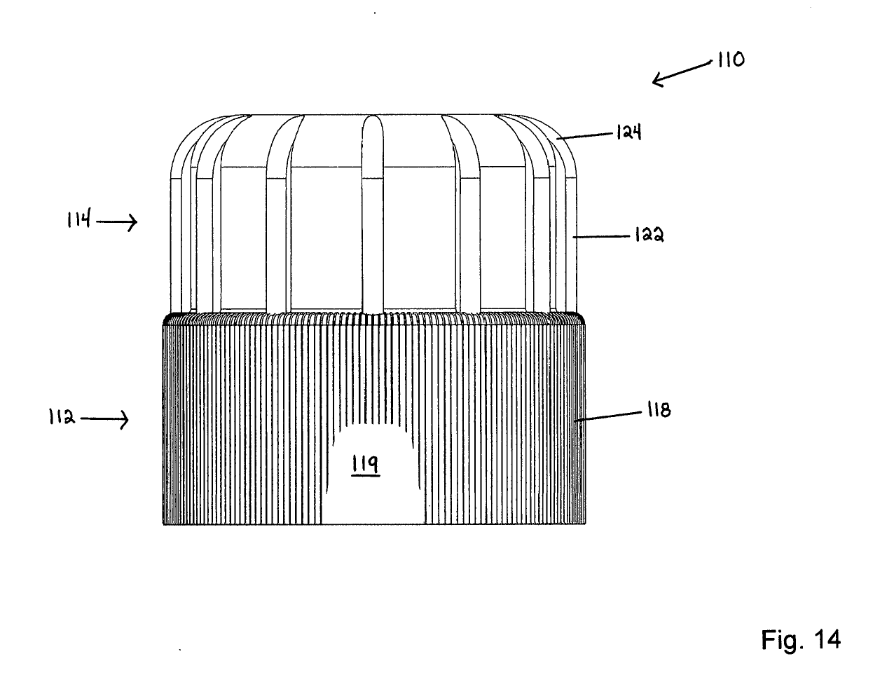

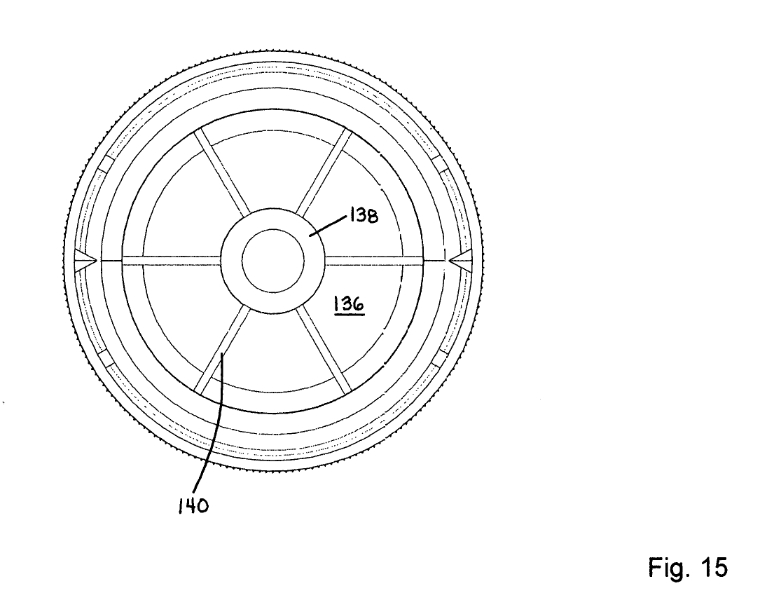

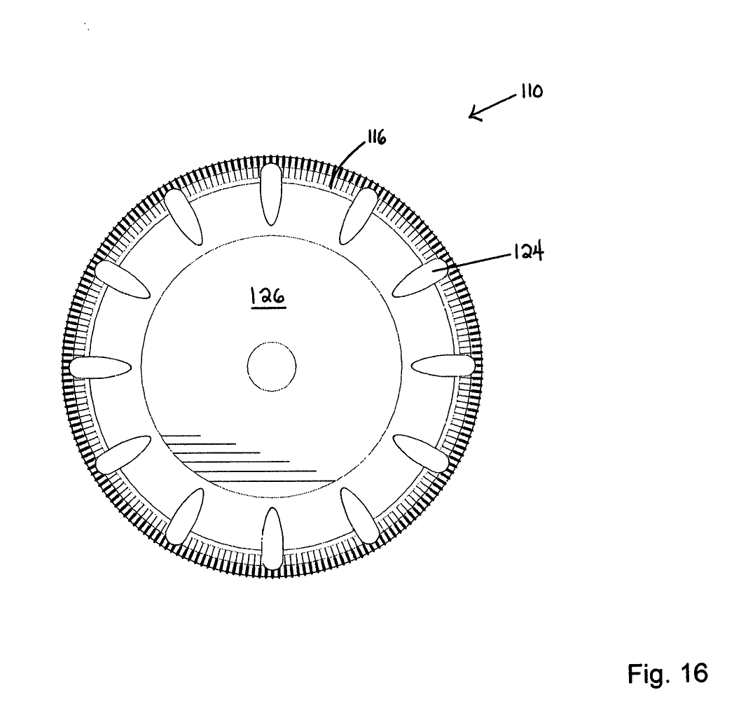

Referring to FIGS. 12 to 16, there is illustrated a preferred embodiment of the cap utilized in the present invention and which cap is generally designated by reference numeral 110.

Cap 110 has a side wall which is comprised of a lower side wall 112 and an upper side wall 114. Lower side wall 112 transitions into upper side wall 114 by means of a ledge or horizontal side wall 116.

On its outer surface, lower side wall 112 is provided with a knurled surface 118 which has a knurled free portion 119. A plurality of ribs 120 are formed on the exterior of upper side wall 112. Each rib 120 has a lower side wall 122 and an upper tapered portion 124. A top wall 126 of cap 112 has a rounded upper side portion 127 on which tapered portion 124 is located.

Interiorly of the cap, there is a lower side wall inner surface 128 and an upper side wall inner surface 130. Interconnecting the inner surfaces 128 and 130 is an interior surface 129 of horizontal side wall 116.

As may be seen in FIG. 12, wedges 134 are formed where lower side wall inner surface 128 meets with the interior of horizontal side wall 116.

Top wall inner surface 136 includes an annular protrusion 138 to seat on the roller and ensure that the same will seal in a leak proof manner with the valve. Reinforcing ribs 140 extend along the interior surface 136.

The arrangement of having ribs extending up in conjunction with the upper tapered portion 124 provides a cap which can be manufactured relatively inexpensively due to less material being required. At the same time, the cap is sufficiently strong to maintain a downward pressure by annular protrusion 138 to seat on the roller. Since these containers are occasionally put in situations where reduced air pressure is present, such as when flying, this is important to provide the leak proof seal.

It will be understood that the above described embodiments are for purposes of illustration only and that changes and modifications may be made thereto without departing from the spirit and scope of the invention.

I claim:

1. A roll-on dispenser comprising:
a container, said container having a neck surrounding a dispensing outlet;
a ball rotatably mounted in and retained in said neck;
a valve seat formed within said neck;
an outer wall of said neck having an upper portion and a lower portion, said upper portion of said outer wall having threads formed thereon, said lower portion of said outer wall having a locking mechanism thereon; and
a cap, said cap having a cap side wall and a cap top wall, said cap side wall having a cap wall upper portion, a cap wall lower portion, screw threads formed on said cap wall upper portion and a locking member formed on said cap wall lower portion, a protrusion formed on an inner surface of said cap top wall, said protrusion engaging said ball when said cap is tightened on said container.

2. The roll-on dispenser of claim 1 wherein said locking mechanism comprises a stop formed on said lower portion of said outer wall, and said locking member on said cap wall lower portion comprises a rib arranged to abut said stop when said cover is screwed on said container, said rib extending completely along said cap wall lower portion, said rib having a tapering height with a smallest height proximate a bottom of said cap wall lower portion.

3. The roll-on dispenser of claim 1 wherein said locking mechanism on said lower portion of said outer wall has a first wall extending outwardly from said lower portion of said outer wall and a second wall extending from a free end of said first wall to merge with said lower portion of said outer wall, said second wall thereby forming a ramp to permit said locking member formed on the lower portion of said cap side wall to ride therealong prior to engaging with said locking member on said lower portion of said outer wall.

4. The roll-on dispenser of claim 1 wherein said cap side wall has a stepped configuration.

5. The roll-on dispenser of claim 4 wherein said protrusion formed on said inner surface of said cap wall has a circular configuration.

6. The roll-on dispenser of claim 1 wherein said cap side wall has a knurled surface.

7. The roll-on dispenser of claim 1 wherein said screw threads formed on said upper portion of said outer wall have a double start.

8. The roll-on dispenser of claim 7 wherein there are provided two screw threads, each of said screw threads extending approximately half of the circumference of said upper portion of said outer wall.

9. The roll-on dispenser of claim 1 wherein said cap side wall and said top wall include indicators showing how to remove said cap.

10. The roll-on dispenser of claim 1 wherein said cap side wall includes interior ribs.
